(19) United States
(12) Patent Application Publication
(10) Pub. No.: US 2014/0277311 A1
(43) Pub. Date: Sep. 18, 2014

(54) IMPLANTABLE MEDICAL LEAD AND METHOD OF MAKING SAME

(71) Applicant: PACESETTER, INC., (US)

(72) Inventors: Keith Victorine, Santa Clarita, CA (US); Sergey Safarevich, Valencia, CA (US); Steven R. Conger, Agua Dulce, CA (US); Serdar Unal, Los Angeles, CA (US); Sean Matthew Desmond, Moorpark, CA (US)

(73) Assignee: PACESETTER, INC., Sylmar, CA (US)

(21) Appl. No.: 13/840,642

(22) Filed: Mar. 15, 2013

(51) Int. Cl. A61N 1/05

(52) U.S. Cl. CPC A61N 1/05
USPC 607/116; 29/825

(57) ABSTRACT

An implantable medical lead includes a longitudinally extending body, an electrical conductor, an electrical component and a weld. The longitudinally extending body includes a distal end, a proximal end, and paddle region near the distal end. The electrical conductor extends through the body between the proximal end and the paddle region. The electrical component is on the paddle region and includes a sacrificial feature defined in a wall of the electrical component. The sacrificial feature includes a region that continues from the wall of the electrical component and a side that is isolated from the wall of the electrical component via a void defined in the wall of the electrical component. The weld is formed at least in part from at least a portion of the sacrificial feature. The weld operably couples the electrical component to the electrical conductor.

### CROSS REFERENCE TO RELATED APPLICATIONS

This application is related to copending U.S. patent application Ser. No. ______, filed ______, and is incorporated herein by reference in its entirety.

### FIELD OF THE INVENTION

The present invention relates to medical apparatus and methods. More specifically, the present invention relates to implantable medical leads and methods of manufacturing such leads.

### BACKGROUND OF THE INVENTION

Implantable pulse generators, such as pacemakers, defibrillators, implantable cardioverter defibrillators (“ICD”) and neurostimulators, provide electrotherapy via implantable medical leads to nerves, such as those nerves found in cardiac tissue, the spinal column, the brain, etc. Electrotherapy is provided in the form of electrical signals, which are generated in the pulse generator and travel via the lead's conductors to the electrotherapy treatment site.

Patients may benefit from electrotherapy treatments to be proposed in the future. However, current conventional lead manufacturing technology has generally limited the extent to which leads can be reduced in size and the elements or features that can be carried on leads.

There is a need in the art for a lead having a configuration that allows the lead to have a reduced size and which is capable of supporting elements or features in a variety of configurations. There is also a need in the art for a method of manufacturing such a lead and manufacturing methods that reduce the cost of such leads.

### BRIEF SUMMARY OF THE INVENTION

An implantable medical lead is disclosed herein. In one embodiment, the lead includes a longitudinally extending body, an electrical conductor, an electrical component and a weld. The longitudinally extending body includes a distal end, a proximal end, and paddle region near the distal end. The electrical conductor extends through the body between the proximal end and the paddle region. The electrical component is on the paddle region and includes a sacrificial feature defined in a wall of the electrical component. The sacrificial feature a region that continues from the wall of the electrical component and a side that is isolated from the wall of the electrical component via a void defined in the wall of the electrical component. The weld is formed at least in part from at least a portion of the sacrificial feature. The weld operably couples the electrical component to the electrical conductor.

In one embodiment, the electrical component includes an electrode. In one embodiment, the electrical component is a strain gage, a pressure sensor, a piezoelectric sensor, an integrated chip, an inductor, or a position tracking sensor.

In one embodiment, the lead further includes a crimp secured to the electrical conductor and the weld is also formed at least in part from at least a portion of the crimp. In one embodiment, the crimp includes a crimp-thru type crimp.

In one embodiment, the sacrificial feature includes a welding tab. In one embodiment, the welding tab is peninsular within the void defined in the wall of the electrical component.

In one embodiment, the electrical component further includes a planar portion including the wall and edges, and the void is defined in the wall between, and spaced away from, the edges. In one embodiment, the electrical component further includes a planar portion including the wall and edges, and the void is defined in the wall at one of the edges.

In one embodiment, the electrical component further includes a plateau and a first riser extending generally perpendicular from a first end of the plateau through a substrate of the paddle region. The plateau serves as an outward facing exposed surface of the electrical component. In one embodiment, the plateau includes the wall and the void is defined in the wall.

In one embodiment, the electrical component further includes a first anchor tab coupled to the plateau via the first riser and extends generally parallel to the plateau. At least a portion of the substrate extends between the plateau and the first anchor tab. In one embodiment, the plateau includes the wall and the void is defined in the wall. In one embodiment, the anchor tab includes the wall and the void is defined in the wall.

In one embodiment, the electrical component further includes a second riser and a second anchor tab. The second riser extends generally perpendicular from a second end of the plateau through a substrate of the paddle region. The second end is generally spaced away from the first end. In one embodiment, the first and second anchor tabs extend towards each other. In one embodiment, the first and second anchor tabs extend away from each other.

A method of assembling an implantable medical lead is also disclosed herein. In one embodiment, the method includes: supporting an electrical component on a paddle region of a lead body, the electrical component including a sacrificial feature defined in a wall of the electrical component, the sacrificial feature including a region that continues from the wall of the electrical component and a side that is isolated from the wall of the electrical component via a void defined in the wall of the electrical component; and welding at least a portion of the sacrificial feature, a resulting weld operably coupling the electrical component to an electrical conductor extending through the lead body.

In one embodiment of the method, the electrical component includes an electrode. In one embodiment of the method, the electrical component includes a strain gage, a pressure sensor, a piezoelectric sensor, an integrated chip, an inductor, or a position tracking sensor.

In one embodiment, the method further includes securing a crimp to the electrical conductor, and also welding at least a portion of the crimp to form at least a part of the resulting weld. For example, the crimp is secured to the electrical conductor via a crimp-thru type crimping process.

In one embodiment of the method, the sacrificial feature includes a welding tab. In one embodiment of the method, prior the welding the at least a portion of the sacrificial feature, the welding tab is peninsular within the void defined in the wall of the electrical component.

In one embodiment of the method, the electrical component further includes a planar portion including the wall and edges, and the void is defined in the wall between, and spaced away from, the edges. In one embodiment of the method, the electrical component further includes a planar portion including the wall and edges, and the void is defined in the wall at one of the edges.

In one embodiment of the method, the electrical component further includes a plateau and a first riser extending generally perpendicular from a first end of the plateau, the method further including causing the first riser to extend through a substrate of the paddle region and causing the plateau to serve as an outward facing exposed surface of the electrical component. In one embodiment of the method, the plateau includes the wall and the void is defined in the wall.

In one embodiment of the method, the electrical component further includes a first anchor tab coupled to the plateau via the first riser, the method further includes causing the first anchor tab to extend generally parallel to the plateau such that at least a portion of the substrate extends between the plateau and the first anchor tab. In one embodiment, the plateau includes the wall and the void is defined in the wall. In one embodiment, the anchor tab includes the wall and the void is defined in the wall.

In one embodiment of the method, the electrical component further includes a second riser and a second anchor tab, the second end being generally spaced away from the first end, the second riser extending generally perpendicular from a second end of the plateau, the method further including causing second riser to extend through the substrate of the paddle region.

In one embodiment, the method further includes causing the first and second anchor tabs to extend towards each other. In one embodiment, the method further includes causing the first and second anchor tabs extend away from each other.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is an isometric view of a paddle style implantable medical lead and a pulse generator for connection thereto.

FIG. 1B is a longitudinal cross section through the paddle style implantable medical lead of FIG. 1A.

FIG. 2A is an isometric view of an electrode employing a peninsular welding tab for welding to a crimp-thru crimp crimped to an electrical conductor extending through the lead body.

FIG. 2B is the same view of the electrode of FIG. 2A, except the electrode has a dual peninsular welding tab configuration.

FIG. 2C is a bottom view of another electrode configuration wherein the peninsular welding tab is defined in an inward projecting anchor tab.

FIG. 2D is the same view of the electrode of FIG. 2C, except the electrode has a peninsular welding tab configuration intersecting a free edge of anchor tab.

FIG. 3A is an enlarged view of the single peninsular welding tab configuration of the electrodes depicted in FIGS. 2A and 2C.

FIG. 3B is an enlarged view of the dual peninsular welding tab configuration of the electrode depicted in FIG. 2B.

FIG. 3C is an enlarged view of an alternative configuration of a single peninsular welding tab.

FIG. 3D is an enlarged view of the peninsular welding tab configuration of the electrode depicted in FIG. 2D.

FIG. 4 is an isometric view of an intermediate manufacturing configuration applicable to anyone of the electrodes depicted in FIGS. 2A-2D, but especially with respect to the embodiment depicted in FIG. 2D.

FIG. 5 is an isometric view of a crimp connector electrically and mechanically coupled to the electrically conductive core of an electrical conductor.

FIGS. 6A-6C are, respectively, top isometric, bottom isometric, and longitudinal cross sectional views of the electrode of FIGS. 2A and 2B electrically and mechanically coupled to the electrical conductor.

FIG. 7 is the same longitudinal cross sectional view as FIG. 6C, except of the electrode of FIGS. 2C and 2D electrically and mechanically coupled to the electrical conductor.

### DETAILED DESCRIPTION

An implantable medical lead 10 is disclosed herein and illustrated in FIG. 1A. In one embodiment, the implantable medical lead 10 is a paddle lead 10 and includes a longitudinally extending body 50, at least one electrical conductor 100, and at least one electrical component 80, such as, for example, an electrode for sensing or pacing, a defibrillation coil, a strain gage, a pressure sensor, a piezoelectric sensor, an integrated chip, an inductor, a position tracking sensor, etc. The body 50 includes a distal end 45 and a proximal end 40. The distal end 45 is in the form of a paddle region 51, and the at least one electrical component 80 is supported on the paddle region 51. For example, the at least one electrical component 80 may be a plurality or array of flat electrodes 80, wherein each flat electrode may be rectangular or have another shape.

As illustrated in FIG. 1B, which is a longitudinal cross section of the distal region of the paddle lead of FIG. 1A, at least one electrical conductor 100 extends through the body 50 between the proximal end 40 and its respective electrical component 80 supported on the distal end 45 and includes a location 110 along its length wherein the electrical component 80 is electrically and mechanically coupled to the electrical conductor 100.

In one embodiment as shown in FIGS. 5-7, the location 110 on the electrical conductor 100 may additionally include a thin-walled, crimp-thru crimp 120 crimped to the location 110 on the conductor 100 to electrically and mechanically couple the crimp 120 to the electrical conductor 100. In other embodiments, the crimp 120 may be a tube 120 or other structure that is welded or otherwise mechanically and electrically coupled to the electrical conductor 100 at the location 110. In being so coupled to the electrical conductor 100 at the location 110, the crimp or tube 120 may extend about at least a portion of an outer circumference of the electrical conductor 100 at the location 110.

To facilitate the welding of the electrical component 80 to the electrical conductor 100 directly or via the intervening crimp or tube 100, the electrical component 80 includes an isolated, sacrificial welding tab 125. Employing welding tabs 125 as disclosed herein in the manufacture of leads 10 offers a number of benefits. First, a successful weld requires less energy when employing the welding tab 125 due to the concentration of the heat on the welding tab 125. Stated differently, by isolating the sacrificial welding tab 125, the heat generated from welding is concentrated in a localized area, thereby reducing the welding heat propagating into the lead body 50 and underlying crimp 120. By concentrating the heat on the welding tab 125, a low energy weld may be performed. Second, employing welding tabs 125 facilitates crimp-thru technology, which reduces the overall size and cost of a lead 10. Third, employing welding tabs 125 facilitates the use of thinner walled crimps, which helps to reduce lead diameter. Fourth, less intimate contact between metal parts prior to welding is required for a consistent and reliable weld when employing welding tabs 125. Fifth, employing welding tabs 125 provides a controlled welding process due to consistent heat transfer in parts subjected to welding because there is a controlled heat sink region, thereby making the welding process and resulting weld more forgiving and less operator dependant. Finally, the welding tab 125 is also more conformal during welding as it allows for greater and more controlled flow of the molten metal between the electrode and crimp sleeve, thereby resulting in more consistent welds to thinner walled crimps and facilitating the downsizing in the diameter of lead bodies. As a result of these benefits, lead manufacturing costs are reduced, smaller diameter lead bodies are facilitated, electrical insulation jackets of electrical conductors 100 are not degraded or otherwise damaged by the welding, and welds are less likely to become contaminated and weak during the welding process.

For a general discussion of an embodiment of a lead 10 employing the above-described tabbed welded connection, reference is made to FIG. 1A, which is an isometric view of the paddle-style implantable medical lead 10 and a pulse generator 15 for connection thereto. The pulse generator 15 may be a pacemaker, defibrillator, ICD or neurostimulator. As indicated in FIG. 1A, the pulse generator 15 includes a can 20, which houses the electrical components of the pulse generator 15, and a header 25. The header is mounted on the can 20 and configured to receive a lead connector end 35 in a lead receiving receptacle 30.

As shown in FIG. 1A, in one embodiment, the lead 10 includes a proximal end 40, a distal end 45 and a tubular body 50 extending between the proximal and distal ends. The proximal end 40 includes a lead connector end 35 including a pin contact 55, a first ring contact 60, a second ring contact 61, which is optional, and sets of spaced-apart radial seals 65. In some embodiments, the lead connector end 35 includes the same or different seals and may include a greater or lesser number of contacts. For example, the lead connector end 35 may be in the form of an IS-1, IS-4, DF-1, etc. configuration. The lead connector end 35 is received in a lead receiving receptacle 30 of the pulse generator 15 such that the seals 65 prevent the ingress of bodily fluids into the respective receptacle 30 and the contacts 55, 60, 61 electrically contact corresponding electrical terminals within the respective receptacle 30.

As illustrated in FIG. 1A, in one embodiment, the lead distal end 45 is in the form of a paddle region 51, and the at least one electrical component 80 is supported on the paddle region 51. For example, the at least one electrical component 80 may be a plurality or array of flat electrodes 80, wherein each flat electrode may be rectangular or have another shape.

As can be understood from FIGS. 1A and 1B, in one embodiment, the electrical components 80 (e.g., various electrodes or groups of electrodes) are in electrical communication with respective electrical contacts on the lead connector end 35, such electrical contacts including the pin contact 55 via a first electrical conductor 100, the first ring contact 60 via a second electrical conductor 100, and the second ring contact 61 via a third electrical conductor 100. In yet other embodiments, other lead components (e.g., additional electrodes, various types of sensors, etc.) mounted on the lead body distal region 45 or other locations on the lead body 50 are in electrical communication with a third ring contact (not shown) similar to the second ring contact 61 via a fourth electrical conductor 100.

Depending on the embodiment, electrical connections in a lead body 50 between a location 110 on an electrical conductor 100 of the lead 10 and the electrical component or device 80 (e.g., an electrode for sensing or pacing, a defibrillation coil, a strain gage, a pressure sensor, an integrated chip, an inductor, a position tracking sensor, etc.) of the lead 10 served by the electrical conductor are accomplished via welding, crimping or a combination of welding and crimping. Crimp-thru technology employing thin-walled crimps or tubes 120 (see FIG. 5) has several useful benefits including facilitating the manufacture of leads 10 having bodies 50 with minimized diameters and reducing manufacturing costs. Crimp-thru technology with thin-walled components 120 allows the thin-walled crimp 120 to be crimped directly through the electrical insulation (e.g., ETFE liner) jacketing the cable conductors 100, which removes a costly pre-ablation process on the cable conductors 100.

Current welding techniques have proven challenging when welding onto thin-walled crimp-thru crimps 120 because the elevated weld energy melts the thin metallic components causing weld penetration to the underlying ETFE insulation, which then vaporizes the ETFE and destroys the weld integrity. To address the issues presented by welding to a thin-walled crimp-through crimp 120, a component 80 (e.g., an electrode for sensing or pacing, a defibrillation coil, a strain gage, a pressure sensor, a piezoelectric sensor, an integrated chip, an inductor, a position tracking sensor, etc.) having a welding tab 125 has been developed and is described in detail below. The welding tab 125 of the component 80 allows for a lower energy weld due to the concentration of the energy on the sacrificial weld tab 125. This low energy weld does not penetrate down to the ETFE insulation and allows for consistent welding to a thin-walled crimp-thru crimp 120.

For a detailed discussion regarding a component 80, such as, for example, an electrode 80, employing the welding tab 125, reference is now made to FIGS. 2A-2D, which is are isometric views of different embodiments of the electrode 80. As shown in FIGS. 2A-2D, the electrode 80 is in the form of a thin-walled rectangular box-like body having a first anchoring tab 130, a second anchoring tab 135, and a raised or offset plateau portion 136 between the two anchoring tab 130, 135. The plateau portion 136 includes a flat electrically active outward surface 140 extending across the plateau portion 136 between the two anchoring tab 130, 135. The plateau portion 136 also includes an inner flat surface 145 on an opposite side of the plateau portion 136 from the outward surface 140. First and second riser members 137, 138 extend perpendicularly between each respective opposed end of the plateau portion 136 and the adjacent anchoring tab 130, 135. The anchoring tabs 130, 135 and electrically active outward surface 140 are generally parallel to each other and perpendicular to the riser members 137, 138.

As illustrated in FIGS. 2A-2B, in some embodiments, the anchoring tab 130, 135 project outwardly away from each other. However, in other embodiments, as indicated in FIGS. 2C-2D, the anchoring tab 130, 135 project inwardly towards each other.

As indicated in FIGS. 2A-2D, each electrode 80 may have a welding tab 125, and the welding tab 125 may have different locations on the electrode depending on the embodiment. For example, in some embodiments as indicated in FIGS. 2A-2B, the welding tab 125 may be defined in the plateau portion 136. However, in other embodiments as depicted in FIGS. 2C-2D, the welding tab 125 may be defined in one of the anchoring tab 130, 135.

As illustrated in FIGS. 2A-2D, the welding tab 125 may have a variety of configurations. For example, as shown in FIGS. 2A and 2C and further detailed in FIG. 3A, the welding tab 125 may be considered to have a peninsula configuration. In other words, as illustrated in FIG. 3A, the welding tab 125 can be said to be defined in a generic wall 147 of the electrode 80 (e.g., a wall 147 forming the plateau portion 136 or anchoring tab 130, 135) so as to extend continuously and uninterrupted from the rest of the wall 147 so as to project into a surrounding space or void 155 defined in and through the wall 147. On account of the peninsular welding tab 125 projecting into the void 155, the welding tab 125 can be considered to include a side or region 160 that extends continuously and uninterrupted from the rest of the wall 147 such that the welding tab inner surface and the welding tab outer surface run continuous and uninterrupted from the inner and outer surfaces of the rest of the wall 147 of the plateau portion 136 or anchoring tab 130, 135 in which the welding tab 125 is defined. Due to the peninsular welding tab 125 projecting into the void 155, the welding tab 125 can be considered to have a free border edge 175 that defines one side of the void 155 and may have multiple side segments 175a-c that define sides of the peninsular welding tab 125 that border the void 155.

As illustrated in FIG. 3A, the void 155 is an opening defined in and through the wall 147, thereby placing the inner and outer (i.e., opposite) surfaces of the wall 147 in communication with each other through the wall 147. The boundaries of the void 155 are defined by the free border edge 175 of the welding tab 125 on one side and another edge 180 of the wall 147 defined by, and across, the void 155 from the free border edge 175. The void 155 may have a horseshoe shape with the peninsular welding tab 125 located between the two side legs or extensions of the horseshoe shape.

The welding tab 125 may be positioned at any angle to match the orientation and shape of the underlying crimp 120. For example, as indicated in FIG. 2A, in one embodiment, the welding tab 125 may be oriented such that its longitudinal axis is parallel relative to the longitudinal axis of the electrode 80. In other embodiments, the welding tab 125 may be oriented such that its longitudinal axis is angled relative to the longitudinal axis of the electrode 80.

Depending on the embodiment, the tab-void configuration may be a single peninsular configuration with a horseshoe shaped void as discussed above with respect to FIGS. 2A, 2C, and 3A or may have a multiple peninsular configuration. For example, in one embodiment as shown in FIG. 2B and further detailed in FIG. 3B, there may be two or more peninsular welding tabs 125 defined by a single void 155. In one embodiment, the there are two welding tabs 125, which are directly opposite each other across the void 155. The welding tabs 125 are configured similar to as described with respect to FIG. 3A, and, since the welding tabs 125 project directly towards each other in an opposed fashion across the void 155, the void 155 can be said to have a H-shaped appearance. The multi-tabbed configuration of FIG. 3B may increase the mechanical strength of a weld to the crimp 120 formed via the multiple welding tabs 125.

The opposed two-tab configuration of FIG. 3B is but one example of a multi-tab configuration. In another multi-tab configuration, one welding tab 125 may be positioned and configured similar to that depicted in FIG. 3A, while the other welding tab 125 may be configured and located as indicated in FIG. 3D below. In other words, one welding tab 125 may be generally centered in the wall 147 of the electrode 80 and the other welding tab 125 may be defined in one of the edges of the wall 147 of the electrode 125.

The electrodes 80 of FIGS. 2A-2D may be formed of a biocompatible metal such as, for example, platinum, platinum-iridium alloy, stainless steel, etc. The welding tab 125 can be manufactured into the electrode 80 via a variety of methods. For example, where the electrode 80 has sufficient thickness and size, the welding tab 125 may be machined into the electrode. Where the electrode 80 is too small or thin-walled for machining, manufacturing methods such as, for example, plunge/wire EDM or laser cutting technology may be employed to define the welding tab 125 in the electrode.

Laser technology is advantageous as it allows platinum parts to be cut into nearly any shape. As a result, laser technology may be used to define in the electrode 80 one or more welding tabs 125 of nearly any shape. For example, a peninsular welding tab 125 may have a shape that is different from the trapezoidal or truncated triangle shape depicted in FIGS. 3A-3B. As depicted in FIG. 3C, which is the same view as FIGS. 3A-3B, except of a welding tab 125 having a different shape, in one embodiment, the peninsular welding tab 125 has a conical base 125a extending from the rest of the wall 147, the conical base 125a transitioning into a circular-shaped free end 125b. Such a shaped welding tab 125 is tailored to take advantage of a circle weld spot. Further, such a shaped welding tab 125 provides a benefit for the operator who can easily target the laser welding beam on the center of the circular-shaped free end 125b of the welding tab 125. Of course, such a shaped welding tab 125 is merely an example of the numerous configurations a welding tab 125 may take.

Depending on how the overall component 80 is to appear in its finished state, the defining of the welding tab 125 may occur at different points in the manufacturing of the component. For example, where the component 80 is an electrode 80 or other similar thin-wall component, the electrode 80 may be stamped and formed into an intermediate shape similar to that depicted in FIG. 4. The welding tab 125 could be defined in the electrode 80 prior to being formed into the intermediate shown in FIG. 4 or subsequent to the forming step, depending on the manufacturing embodiment employed.

Rather than being positioned in the center of the plateau portion 136 of the electrode 80 as depicted in FIGS. 2A-2B, the welding tab 125 may be located in a variety of other locations on the ring electrode 80. For example, as illustrated in FIGS. 2C-2D, the welding tab 125 can be defined in edge of the anchor tab 130, 135. The welding tab 125 can still be seen to have a peninsular shape. As indicated in FIG. 3D, the peninsular shaped welding tab 125 defined in an edge of the wall 147 may be substantially rectangular with square corners. However, in other embodiments, the peninsular welding tab 125 may be generally rectangular with rounded corners or the welding tab 125 may employ the above-described trapezoidal shape or other shapes.

As illustrated in FIG. 5, which is an isometric view of a conductor 100 employing a crimp 120, the conductor 100 has an electrically conductive core 200, an electrically insulating jacket 205, and a crimp electrically and mechanically coupled to the conductive core 200. The crimp 120 may be in the form of a crimp sleeve or slug attached to a distal end of the conductor 100 and, more specifically, electrically and mechanically coupled to the electrically conductive core 200 of the conductor 100.

For a discussion of a manufacturing method used to electrically and mechanically couple the electrode 80 of FIGS. 2A and 2B to an electrical conductor 100 extending through the lead body 50, reference is made to FIGS. 4, 5 and 6A-6C. FIGS. 6A-6C are, respectively, top isometric, bottom isometric, and longitudinal cross sectional views of the electrode 80 electrically and mechanically coupled to the electrical conductor 100.

As discussed above with respect to FIG. 4, the electrode 80 may be stamped to be configured such that the risers 137, 138 are generally a continuous planar member with the anchor tabs 130, 135, which are perpendicular to the plateau portion 136. As can be understood from FIG. 6C, the intermediate configuration of the electrode 80 as depicted in FIG. 4 is inserted through the substrate 210 of the paddle portion 51 of the lead body 50 such that the anchor tabs 130, 135 extend through and fold about the inner surface 215 of the substrate 210. As depicted in FIG. 5, a crimp connector 120 (e.g., crimp sleeve or slug) that is mechanically and electrically coupled to the distal end of the electrically conductive core 200 of the electrical conductor 100 in a region of the conductor 100 wherein the insulation jacket 205 is removed to expose the core 200. As illustrated in FIGS. 6A-6C, the crimp connector 120 is mechanically and electrically coupled to the weld tab 125 via a weld administered in the region occupied by the weld tab 125. Further, the anchor tabs 130, 135 extend oppositely from each other away from the plateau portion 136 of the electrode 80.

As can be understood from FIG. 7, which is a longitudinal cross section of the electrode of FIGS. 2C and 2D mounted in the substrate 210, a manufacturing method similar to that as described above with respect to FIGS. 4-6C can be employed, except the anchor tabs 130, 135 extend towards each other and underneath the plateau portion 136 of the electrode 80.

For the various embodiments described above, the resulting weld is robust. Further, the substrate and the polymer layers of the lead body 50 and the electrical insulation jacket of the conductor 100 have not been adversely impacted by the welding process. The configuration of the welding tab 125 results in weld nugget that is thicker and stronger than would otherwise be possible with such low welding energy as employed in making the weld nugget.

While the above-described embodiments are given in the context of the component 80 being an electrode 80, it should be noted that the above-described welding tab configurations and associated teachings may be applied to other components 80 including, for example, shock coils or other components that weld in a similar fashion to electrodes. The welding tab configurations and associated teachings disclosed herein may also apply for other termination methods such as, for example, making electromechanical connections to sensors.

As can be understood from FIGS. 3A-3D, the welding tab 125 is part of the wall 147 of the electrode 80 via one welding tab side or region 160 being an extension of the rest of the wall 140 of the electrode 80. However, the other three welding tab sides 175a-c are isolated from the rest of the wall 147. As a result, the welding tab 125 can be used as an isolated, sacrificial welding tab 125 offering certain benefits. For example, as can be understood from FIGS. 3A-3D, 6A-6C and 7A-7C, the isolated, sacrificial welding tab 125 defined in the wall 147 of the electrode 80 allows for concentration of the heat from welding in a localized area. As a result, more molten metal is generated for fusion between the electrode 80 and the underlying crimp 120. Specifically, during welding, the laser energy melts the welding tab 125, thereby generating a relatively large welding pool, which then fills the gap between the components (i.e., the electrode 80 and the crimp 120) and fuses them together. The use of the isolated, sacrificial welding tab 125 also allows for a low energy weld with less welding heat propagating into the lead body 50, substrate 210, underlying crimp 120 and conductor 100.

Although the present invention has been described with reference to preferred embodiments, persons skilled in the art will recognize that changes may be made in form and detail without departing from the spirit and scope of the invention.

What is claimed is:

1. An implantable medical lead comprising:
a longitudinally extending body comprising a distal end, a proximal end, and paddle region near the distal end;
an electrical conductor extending through the body between the proximal end and the paddle region;
an electrical component on the paddle region and comprising a sacrificial feature defined in a wall of the electrical component, the sacrificial feature comprising a region that continues from the wall of the electrical component and a side that is isolated from the wall of the electrical component via a void defined in the wall of the electrical component; and
a weld formed at least in part from at least a portion of the sacrificial feature, the weld operably coupling the electrical component to the electrical conductor.

2. The lead of claim 1, wherein the electrical component comprises an electrode.

3. The lead of claim 1, wherein the electrical component comprises a strain gage, a pressure sensor, a piezoelectric sensor, an integrated chip, an inductor, or a position tracking sensor.

4. The lead of claim 1, further comprising a crimp secured to the electrical conductor and the weld is also formed at least in part from at least a portion of the crimp.

5. The lead of claim 4, wherein the crimp comprises a crimp-thru type crimp.

6. The lead of claim 1, wherein the sacrificial feature comprises a welding tab.

7. The lead of claim 6, wherein the welding tab is peninsular within the void defined in the wall of the electrical component.

8. The lead of claim 1, wherein the electrical component further comprises a planar portion comprising the wall and edges, and the void is defined in the wall between, and spaced away from, the edges.

9. The lead of claim 1, wherein the electrical component further comprises a planar portion comprising the wall and edges, and the void is defined in the wall at one of the edges.

10. The lead of claim 1, wherein the electrical component further comprises a plateau and a first riser extending generally perpendicular from a first end of the plateau through a substrate of the paddle region, the plateau serving as an outward facing exposed surface of the electrical component.

11. The lead of claim 10, wherein the plateau comprises the wall and the void is defined in the wall.

12. The lead of claim 10, wherein the electrical component further comprises a first anchor tab coupled to the plateau via the first riser and extending generally parallel to the plateau, at least a portion of the substrate extending between the plateau and the first anchor tab.

13. The lead of claim 12, wherein the plateau comprises the wall and the void is defined in the wall.

14. The lead of claim 12, wherein the anchor tab comprises the wall and the void is defined in the wall.

15. The lead of claim 14, wherein the electrical component further comprises a second riser and a second anchor tab, the second riser extending generally perpendicular from a second end of the plateau through a substrate of the paddle region, the second end being generally spaced away from the first end.

16. The lead of claim 15, wherein the first and second anchor tabs extend towards each other.

17. The lead of claim 15, wherein the first and second anchor tabs extend away from each other.

18. A method of assembling an implantable medical lead, the method comprising:
supporting an electrical component on a paddle region of a lead body, the electrical component comprising a sacrificial feature defined in a wall of the electrical component, the sacrificial feature comprising a region that continues from the wall of the electrical component and a side that is isolated from the wall of the electrical component via a void defined in the wall of the electrical component; and
welding at least a portion of the sacrificial feature, a resulting weld operably coupling the electrical component to an electrical conductor extending through the lead body.

19. The method of claim 18, wherein the electrical component comprises an electrode.

20. The method of claim 18, wherein the electrical component comprises a strain gage, a pressure sensor, a piezoelectric sensor, an integrated chip, an inductor, or a position tracking sensor.

21. The method of claim 18, further comprising securing a crimp to the electrical conductor, and also welding at least a portion of the crimp to form at least a part of the resulting weld.

22. The method of claim 21, wherein the crimp is secured to the electrical conductor via a crimp-thru type crimping process.

23. The method of claim 18, wherein the sacrificial feature comprises a welding tab.

24. The method of claim 23, wherein, prior the welding the at least a portion of the sacrificial feature, the welding tab is peninsular within the void defined in the wall of the electrical component.

25. The method of claim 18, wherein the electrical component further comprises a planar portion comprising the wall and edges, and the void is defined in the wall between, and spaced away from, the edges.

26. The method of claim 18, wherein the electrical component further comprises a planar portion comprising the wall and edges, and the void is defined in the wall at one of the edges.

27. The method of claim 18, wherein the electrical component further comprises a plateau and a first riser extending generally perpendicular from a first end of the plateau, the method further comprising causing the first riser to extend through a substrate of the paddle region and causing the plateau to serve as an outward facing exposed surface of the electrical component.

28. The method of claim 27, wherein the plateau comprises the wall and the void is defined in the wall.

29. The method of claim 27, wherein the electrical component further comprises a first anchor tab coupled to the plateau via the first riser, the method further comprising causing the first anchor tab to extend generally parallel to the plateau such that at least a portion of the substrate extends between the plateau and the first anchor tab.

30. The method of claim 29, wherein the plateau comprises the wall and the void is defined in the wall.

31. The method of claim 29, wherein the anchor tab comprises the wall and the void is defined in the wall.

32. The method of claim 29, wherein the electrical component further comprises a second riser and a second anchor tab, the second end being generally spaced away from the first end, the second riser extending generally perpendicular from a second end of the plateau, the method further comprising causing second riser to extend through the substrate of the paddle region.

33. The method of claim 32, further comprising causing the first and second anchor tabs to extend towards each other.

34. The method of claim 32, further comprising causing the first and second anchor tabs extend away from each other.
